SEQUENCE LISTING (1 sequence)

SEQ ID NO: 1 - DNA, 20 bp, Artificial Sequence
  Synthetic Sequence
tccggaaagttcctagctca

(19) United States
(12) Patent Application Publication
(10) Pub. No.: US 2022/0091104 A1
(43) Pub. Date: Mar. 24, 2022

(54) ANALYSIS OF POLYMORPHISMS IN SIRP-ALPHA FOR EVALUATING RESPONSE TO IMMUNOTHERAPY

(71) Applicant: The Board Of Trustees Of The Leland Stanford Junior University, Stanford, CA (US)

(72) Inventors: Ying Ying Yiu, Concord, CA (US); Michal Caspi Tal, Cupertino, CA (US); Amira Barkal, Stanford, CA (US); Hanna Maria Ollila, Palo Alto, CA (US); Irving L. Weissman, Stanford, CA (US)

(21) Appl. No.: 17/276,052

(22) PCT Filed: Sep. 11, 2019
(86) PCT No.: PCT/US19/50650

§ 371 Date: Mar. 12, 2021

Related U.S. Application Data
(60) Provisional application No. 62/738,221, filed on Sep. 28, 2018.

(51) Int. Cl. G01N 33/50; C12Q 1/6876; G01N 33/574; G01N 33/68

(52) U.S. Cl. CPC G01N 33/5091; C12Q 1/6876; C12Q 2600/158; G01N 33/6872; G01N 33/574

(57) ABSTRACT

Methods and kits are provided for determining whether an individual is responsive to therapeutic CD47 blockade. Specifically, the disclosure provides methods of determining whether a cell or cell population is responsive to therapeutic CD47 blockade, the method comprising: assaying a cell sample from an individual to determine (a) the level of expression of signal regulatory protein alpha (SIRPalpha) on phagocytic immune cells, or (b) the genotype of the individual at a nucleotide polymorphism (SNP) associated with SIRPalpha expression. Further disclosed are SNPs associated with SIRPalpha expression.

### CROSS REFERENCE

This application claims the benefit of U.S. Provisional Application No. 62/738,221, filed Sep. 28, 2018, which is incorporated herein by reference in its entirety.

### BACKGROUND

In multiple preclinical and clinical studies, CD47 has been identified as an anti-phagocytic signal that is highly expressed on human primary cancers. Binding of CD47 to SIRPα, an inhibitory receptor on macrophages, counteracts “eat me” signals and resultantly blocks phagocytosis. Agents that block the binding of CD47 to its inhibitory SIRPα receptor on increases phagocytosis of the CD47-expressing cells by macrophages and other phagocytic cells. Additionally, the phagocytosis of cancer cells or infected cells by macrophages enables presentation of intracellular proteins as peptides by MHC-I surfaces molecules to effector.

Agents that block the interaction of SIRPα and CD47 have the potential to enable the patient's immune system to identify and destroy primary or metastatic cancer cells. However, the observed differential response of cancer cells to anti-CD47 antibody treatment may stratify patients with respect to response patterns. The invention addresses this issue.

Methods of treatment with therapeutic CD47 blockade are disclosed, inter alia, in any of U.S. Pat. Nos. 9,771,428; 9,623,079; 8,562,997; 9,399,682; 9,493,575; 9,624,305; 9,605,076; 9,611,329; 9,765,143; 8,758,750; 9,017,675; 9,382,320; 10,064,925, each herein specifically incorporated by reference.

### SUMMARY

Methods are provided for determining the responsiveness of an individual to therapeutic CD47 blockade, in which therapy the individual is treated with an agent that blocks the interaction of CD47 and SIRPα in order to enhance phagocytosis of CD47 over-expressing cells, such as cancer cells, pathogen-infected cells, etc. It has been found that individuals vary in basal and induced levels of SIRPα expression by immune cells, e.g. dendritic cells, monocytes, macrophages and T cells, including engineered CAR T cells, etc. It is further shown that differences in SIRPα expression are associated with responsiveness to therapeutic CD47 blockade, where cells that express higher SIRPα levels are less responsive to the blockade. Genotypic analysis has identified SNP polymorphisms that predict SIRPα expression levels, which analysis is useful in identifying individuals for treatment.

A DNA sample from an individual is assayed to determine the genotype with respect to polymorphisms that affect expression of SIRPα. In some embodiments the polymorphism is linked to the SNP rs4813322. In some embodiments the polymorphism is SNP rs4813322. An individual may be genotyped as homozygous A/A; homozygous G/G or heterozygous A/G at SNP rs4813322. Presence of one or two A alleles at SNP rs4813322 indicates higher SIRPα expression, and decreased responsiveness to CD47 blockade, where homozygous AA individuals are indicated to have highest levels of SIRPα expression. Genotyping results are optionally confirmed with a direct measurement of SIRPα expression levels in a relevant cell population, e.g. monocytes, macrophages, dendritic cells, etc. In other embodiments an individual is genotyped with a polymorphism set forth in Table 1 or in FIG. 4, for the presence of a polymorphism linked to altered SIRPα expression.

In some embodiments, an individual determined to be of a responsive type is treated with an anti-CD47 agent, i.e. by therapeutic CD47 blockade. In some embodiments, the methods of the invention find use in determining whether to continue or alter therapy, where an individual responsive to CD47 blockade may be treated with the same. In some such embodiments, the individual is being treated with an anti-CD47 agent for cancer. In other embodiments the individual is being treated with an anti-CD47 agent for infection, particularly with an intracellular pathogen.

An anti-CD47 agent for use in the methods of the invention interferes with binding between CD47 present on a target cell, including without limitation a cancer cell, a cell infected with an intracellular pathogen, a stem cell, etc., to SIRPα present on an immune cell. Generally both such cells are present in the individual being treated. Such methods, in the presence of a positive signal that elicits immune cell effector function, can increase phagocytosis or cytotoxicity of the target cell. The subject methods can be used to stratify individuals for treatment, including for example stratification of individuals suitable for clinical trials involving CD47 blockade. Suitable anti-CD47 agents include soluble SIRPα-polypeptides; soluble CD47; anti-CD47 antibodies, anti-SIRPα antibodies, small molecules, and the like, where the term antibodies encompasses antibody fragments and variants thereof, as known in the art.

In some embodiments a kit is provided for practicing the methods described herein, where the kit contains reagents suitable for genotypic analysis, e.g. primers for detecting by sequencing or hybridization the genotype of SNPs, which may include without limitation rs4813322 or the SNP set forth in Table 1 or in FIG. 4. Kits may further comprise buffers, enzymes, and the like for use in genotyping; and instructions for use. The kit reagents may be provided as an in vitro diagnostic (IVD) device for use as a companion diagnostic for therapeutic CD47 blockade, and may utilize next generation sequencing (NGS); amplification based assays, hybridization assays, and the like.

### BRIEF DESCRIPTION OF THE DRAWINGS

The invention is best understood from the following detailed description when read in conjunction with the accompanying drawings. On the contrary, the dimensions of the various features are arbitrarily expanded or reduced for clarity. Included in the drawings are the following figures.

FIG. 1A-1B. Individual variation in basal level and induced SIRPα expression. (FIG. 1A) PBMCs isolated from blood samples from individual donors were stained for expression of SIRPα and markers that identified dendritic cells (identified as cDC2, which cells are CD14-HLA-DR⁺ CD141^{int} CD11c⁺) and monocytes (identified as CD14⁺ cells). The data show variability of SIRPα expression, particularly in the monocyte subset. Shown in (FIG. 1B), PBMCs from each donor were pulsed with CFSE and plated into wells coated with PBS (control) or anti-human CD3/anti-human CD28 antibodies (activated). Cells were analyzed by FACS on day 4 for SIRPα expression. The data show variability in the number of SIRPα positive activated T cells.

FIG. 2. SNP rs4813322 is predicted to impact PU.1 binding in SIRPα enhancer. PU.1/SPI1 is an important transcription factor for myeloid cells, binding of which is affected by the polymorphism in SNP rs4813322. Distribution of the genotypes of the 12 donors from FIG. 1 and representative Sanger Sequencing traces for each genotype is shown.

FIG. 3A-3B. Basal expression of SIRPα varies with SNP rs4813322 genotype. (FIG. 3A) Shown is the basal level of expression of SIRPα in the indicated cell populations. The rs4813322 genotype is closely associated with the level of SIRPα expression on cDC2s and CD14+ cells. Individuals homozygous AA are seen to have the highest expression; heterozygote have intermediate expression and people homozygous GG have the lowest SIRPα expression. (FIG. 3B) Primary human monocyte-derived macrophages were analyzed by FACS for SIRPα expression. Expression is correlated with SNP rs4813322 genotype.

FIG. 4. Query of SNP rs4813322 using HaploReg v4.1. SNP rs4813322 is in linkage disequilibrium (LD), defined by r²>0.8, with the set of SNPs shown in the figure, including rs72620874.

FIG. 5. Complete Extracellular Structure of Human SIRPα (Hatherley et al. J. Biol. Chem. 284:26613-26619 (2009). The extracellular region of SIRPα is visualized using iCn3D from NCBI's Molecular Modeling Database (MMDB). CD47 contact residues are highlighted. SNP rs72620874, which is a missense SNP in LD with rs4813322, lies in a region of the SIRPα protein that is proximal to the CD47 binding site.

FIG. 6. Effect of SNP rs4813322 genotype on phagocytosis by human monocyte-derived macrophages. Target cells expressing CD47 were treated with an antibody that binds to CD47 (5F9-G4) and co-incubated with phagocytic human monocyte-derived macrophages. The level of phagocytosis was determined by flow cytometry. It is shown that the response of macrophages to anti-CD47 blockade correlates with SIRPα expression, where higher SIRPα expression (found in individuals with SNP rs4813322 AA genotype) had a poorer response to anti-CD47 blockade.

FIG. 7. Primary human T cells from seven healthy donors were activated with anti-CD3/CD28 beads and retrovirally transduced with CD19.28z CAR and grown in vitro. Cells were analyzed by FACS for SIRPα expression. The level of SIRPα upregulation by CD8+ CAR T cells varies between donors (See table). Cytotoxicity assays were used to evaluate CD19-CAR T cell cytolytic ability. GFP+ Nalm6 cell line was used as target cells and co-incubated with CAR T cells at a 1:1 ratio. Isotype control antibody (circles) or KWAR, an anti-SIRPα antibody (triangles), was added and target cell GFP signal was monitored for 120 hours. Treatment with KWAR improved CAR T cell killing in donors who have higher percentage of CD8+ T cells expressing SIRPα.

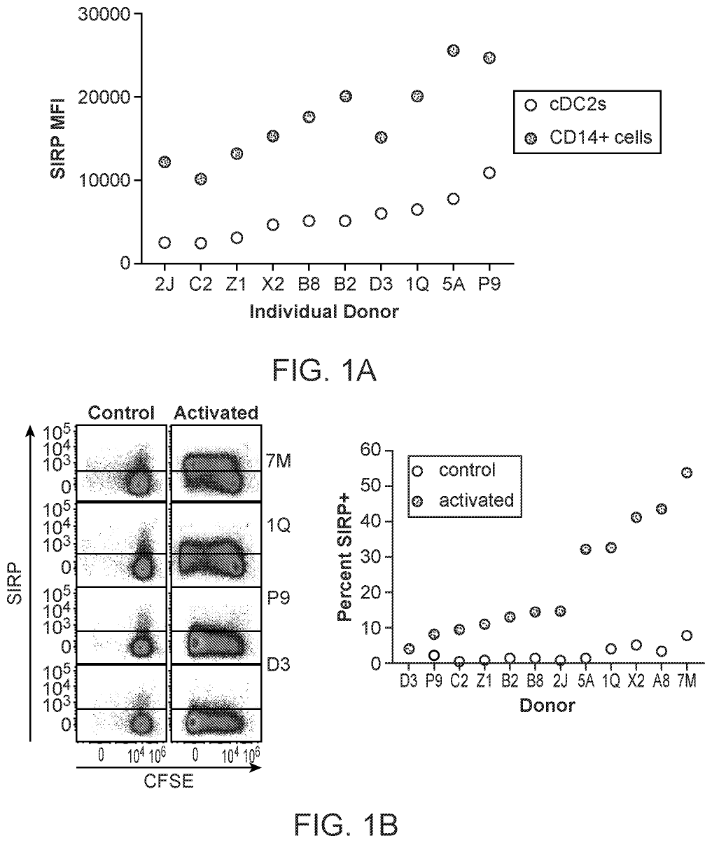

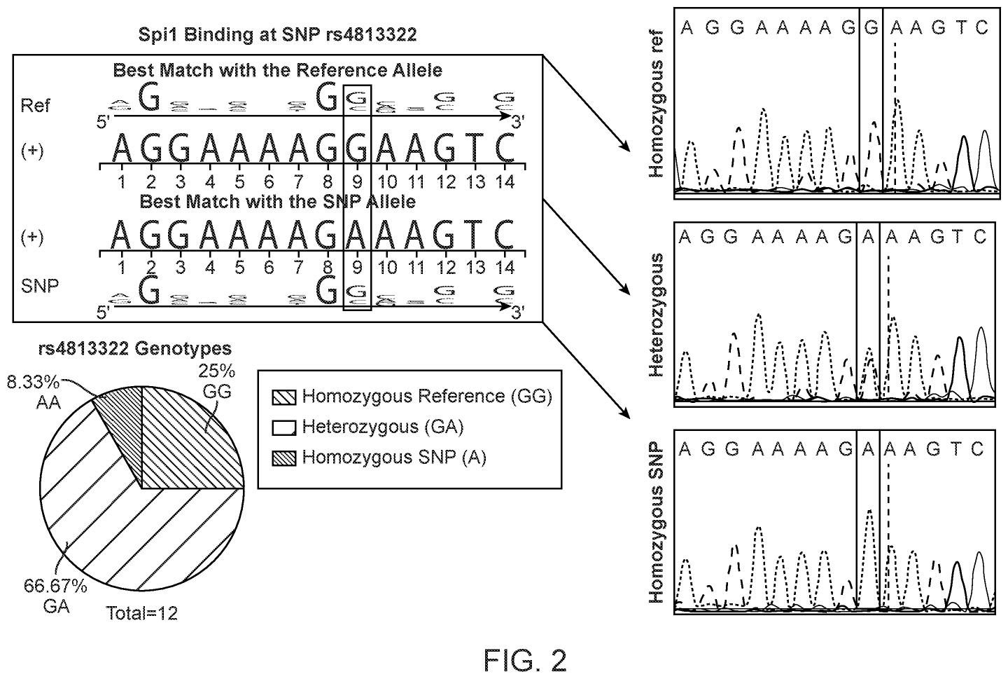

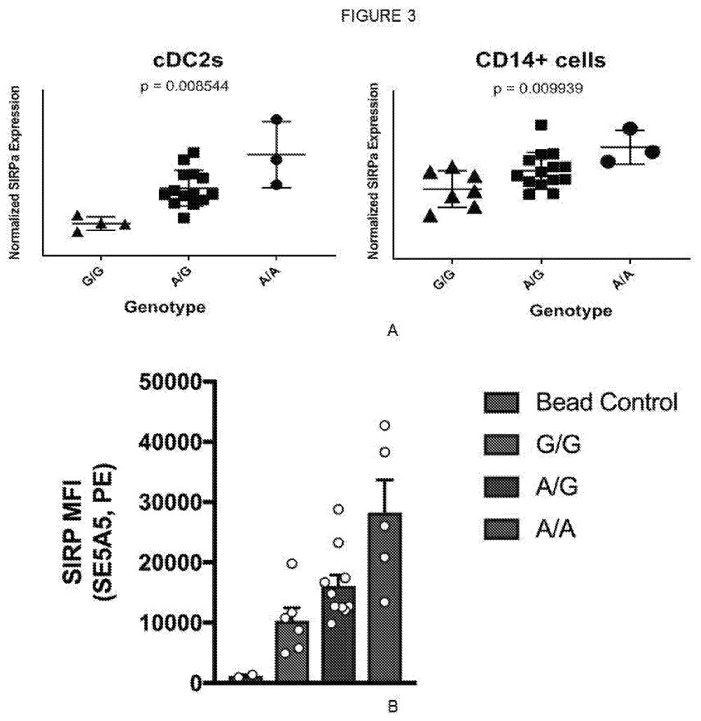

### DETAILED DESCRIPTION OF EMBODIMENTS

The present invention relates to methods of determining whether a cell or population of cells is responsive to therapeutic CD47 blockade, the methods comprising genotyping for polymorphisms associated with the level of expression of SIRPα in immune cells. Also provided are kits and companion diagnostics performing the methods.

Before the present methods and kits are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

SIRPα1 (PTPNS1, SHPS1), is a transmembrane glycoprotein, expressed primarily on myeloid and neuronal cells. SIRPα interacts with the widely distributed membrane protein CD47. In humans, the SIRPα protein is found in two major forms. One form, the variant 1 or V1 form, has the amino acid sequence set out as NCBI RefSeq NP_542970.1 (residues 27-504 constitute the mature form). Another form, the variant 2 or V2 form, differs by 13 amino acids and has the amino acid sequence set out in GenBank as CAA71403.1 (residues 30-504 constitute the mature form). These two forms of SIRPα constitute about 80% of the forms of SIRPα present in humans, and both are embraced herein by the term “human SIRPα”. Also embraced by the term “human SIRPα” are the minor forms thereof that are endogenous to humans and have the same property of triggering signal transduction through CD47 upon binding thereto. Sequences of human SIRPα variants may be accessed through public databases, including Genbank accession numbers: ref|NP_542970.1; gb|EAX10606.1; ref|XP_005260726.1; gb|EAX10606.1; XP_005260726.1; gb|EAX10611.1; gb|EAX10609.1; dbj|JBAA12974.1; gb|AAH26692.1; ref|XP_011527475.1. See, for example Lee et al. (2007) J. Immunol. 179(11):7741-7750; herein specifically incorporated by reference.

SNP rs4813322 is located in an intron just before exon 2 of the SIRPA gene. This region is predicted to be a binding site for PU.1(SPI1) transcription factor; and the nucleotide change at rs4813322 (A/G) is predicted to influence PU.1 binding. SNP rs4813322 may directly influence expression of SIRPα. PU.1 is a pioneer transcription factor (TF) and regions that pioneer TFs bind are usually enhancer regions.

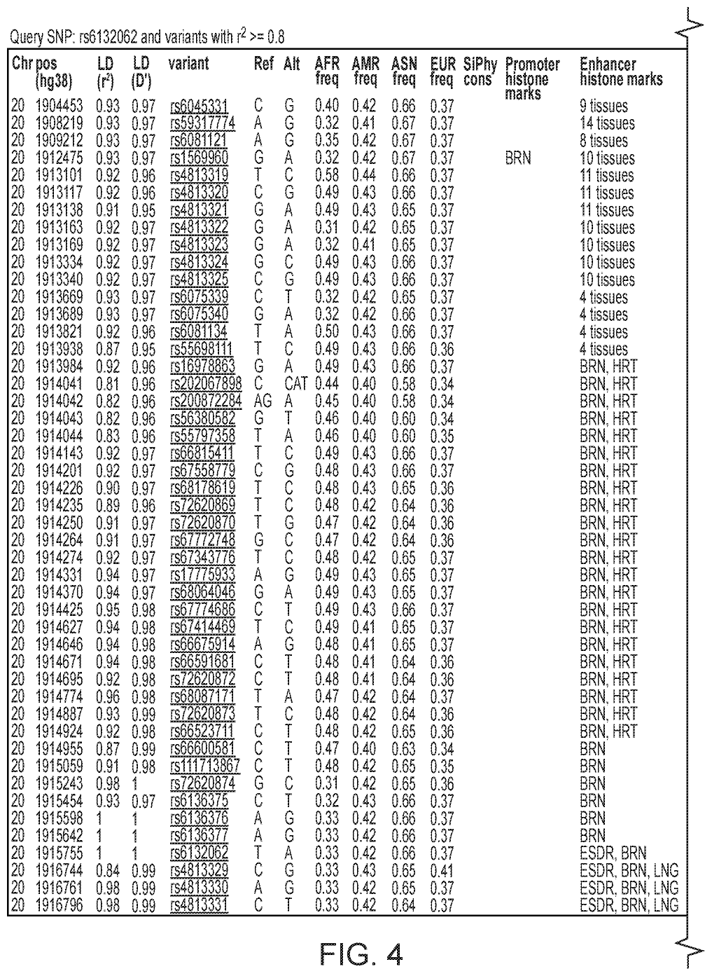

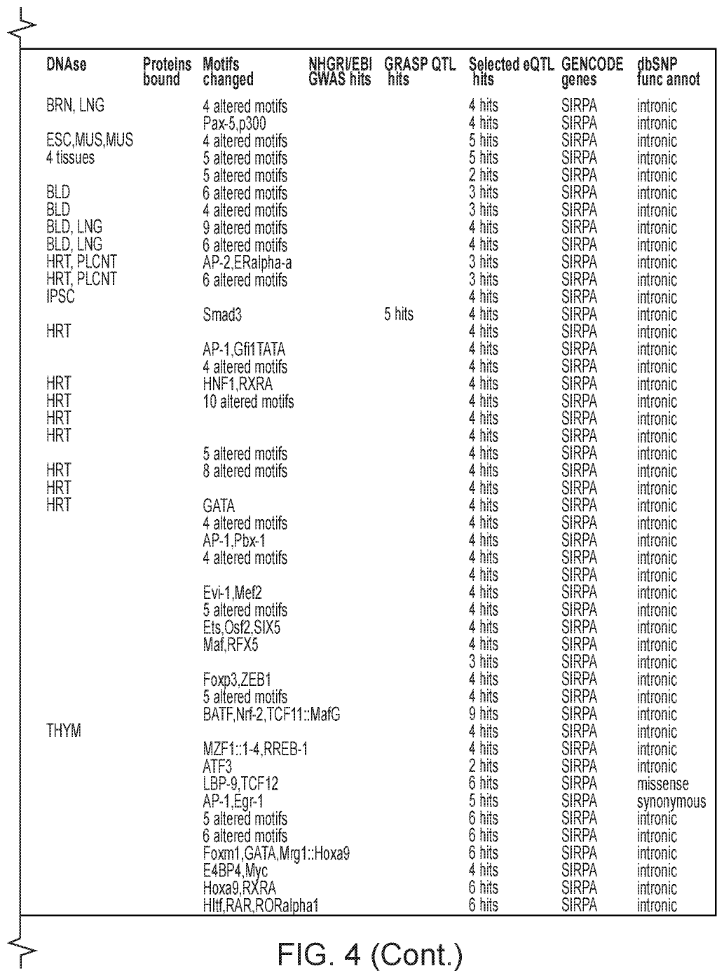

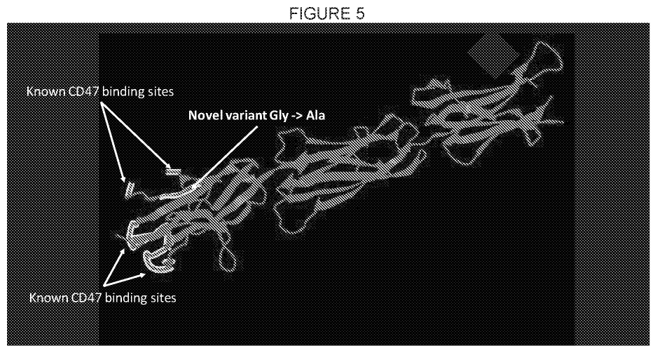

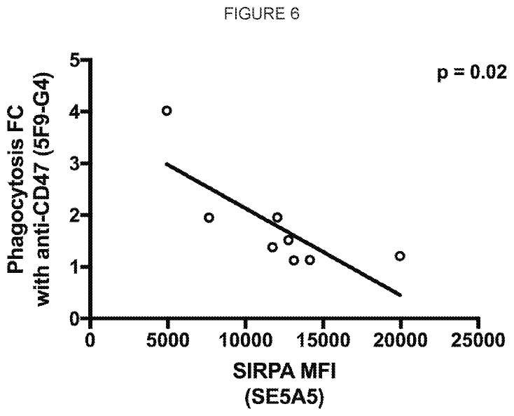

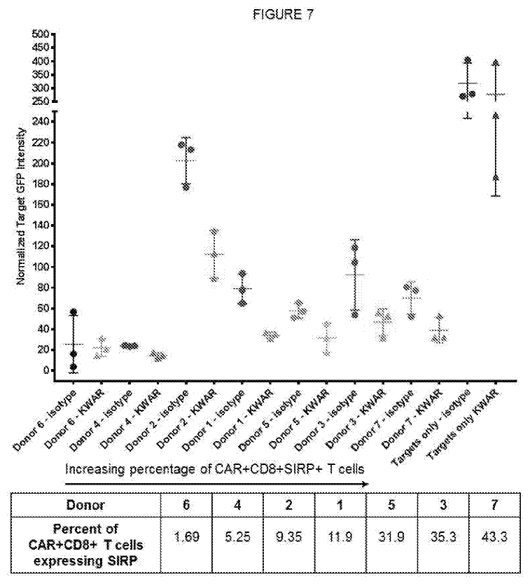

For genotyping purposes linked polymorphisms may also or alternatively be evaluated, where such linked SNPs include, without limitations, those listed in FIG. 4, including rs72620874. Unlinked SNPs that may be evaluated include those set forth in Table 1. Haplotypes are a combination of various genetic markers having particular alleles at polymorphic sites. The haplotype can comprise a combination of various genetic markers, therefore, detecting haplotypes can be accomplished by methods known in the art for detecting sequences at polymorphic sites. Certain methods of identifying relevant markers and SNPs include the use of linkage disequilibrium (LD) and/or LOD scores. The frequencies of haplotypes in the patient and the control groups can be estimated using an expectation-maximization algorithm (Dempster et al., J. R. Stat. Soc. B, 39:1-38 (1977)). An implementation of this algorithm that can handle missing genotypes and uncertainty with the phase can be used. In a preferred embodiment, a p-value of <0.05 is indicative of a significant marker and/or haplotype association.

LD between pairs of markers can be calculated using the standard definition of D′ and R² (Lewontin, R., Genetics 49:49-67 (1964); Hill, W. G. & Robertson, A. Theor. Appl. Genet. 22:226-231 (1968)). Using NEMO, frequencies of the two marker allele combinations are estimated by maximum likelihood and deviation from linkage equilibrium is evaluated by a likelihood ratio test. The definitions of D′ and R² are extended to include microsatellites by averaging over the values for all possible allele combination of the two markers weighted by the marginal allele probabilities. When plotting all marker combinations to elucidate the LD structure in a particular region, we plot D′ in the upper left corner and the p-value in the lower right corner. In the LD plots the markers can be plotted equidistant rather than according to their physical location, if desired.

In certain embodiments, marker and haplotype analysis involves defining a candidate susceptibility locus based on “haplotype blocks” (also called “LD blocks”). It has been reported that portions of the human genome can be broken into series of discrete haplotype blocks containing a few common haplotypes; for these blocks, linkage disequilibrium data provided little evidence indicating recombination (see, e.g., Wall, J. D. and Pritchard, J. K., Nature Reviews Genetics 4:587-597 (2003); Daly, M. et al., Nature Genet. 29:229-232 (2001); Gabriel, S. B. et al., Science 296:2225-2229 (2002); Patil, N. et al., Science 294:1719-1723 (2001); Dawson, E. et al., Nature 418:544-548 (2002); Phillips, M. S. et al., Nature Genet. 33:382-387 (2003)). There are two main methods for defining these haplotype blocks: blocks can be defined as regions of DNA that have limited haplotype diversity (see, e.g., Daly, M. et al., Nature Genet. 29:229-232 (2001): Patil, N. et al., Science 294:1719-1723 (2001); Dawson, E. et al., Nature 418:544-548 (2002); Zhang, K. et al., Proc. Natl. Acad. Sci. USA 99:7335-7339 (2002)), or as regions between transition zones having extensive historical recombination, identified using linkage disequilibrium (see, e.g., Gabriel, S. B. et al., Science 296:2225-2229 (2002); Phillips, M. S. et al., Nature Genet. 33:382-387 (2003); Wang, N. et al., Am. J. Hum. Genet. 71:1227-1234 (2002); Stumpf, M. P., and Goldstein, D. B., Curr. Biol. 13:1-8 (2003)). As used herein, the terms “haplotype block” or “LD block” includes blocks defined by either characteristic.

Representative methods for identification of haplotype blocks are set forth, for example, in U.S. Published Patent Application Nos. 20030099964, 20030170665, 20040023237 and 20040146870. Haplotype blocks can be used readily to map associations between phenotype and haplotype status. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

Biological sample. The term “sample” with respect to an individual encompasses blood and other liquid samples of biological origin, solid tissue samples such as a biopsy specimen or tissue cultures or cells derived or isolated therefrom and the progeny thereof. The definition also includes samples that have been manipulated in any way after their procurement, such as by treatment with reagents; washed; or enrichment for certain cell populations, such as cancer cells. The definition also includes samples that have been enriched for particular types of molecules, e.g., nucleic acids, polypeptides, etc.

DNA samples, e.g. samples useful in genotyping, are readily obtained from any nucleated cells of an individual, e.g. hair follicles, cheek swabs, white blood cells, etc., as known in the art.

The term “biological sample” encompasses a clinical sample. The types of “biological samples” include, but are not limited to: tissue obtained by surgical resection, tissue obtained by biopsy, cells in culture, cell supernatants, cell lysates, tissue samples, organs, bone marrow, blood, plasma, serum, fine needle aspirate, lymph node aspirate, cystic aspirate, a paracentesis sample, a thoracentesis sample, and the like.

Obtaining and assaying a sample. The term “assaying” is used herein to include the physical steps of manipulating a biological sample to generate data related to the sample. As will be readily understood by one of ordinary skill in the art, a biological sample must be “obtained” prior to assaying the sample. Thus, the term “assaying” implies that the sample has been obtained. The terms “obtained” or “obtaining” as used herein encompass the act of receiving an extracted or isolated biological sample. For example, a testing facility can “obtain” a biological sample in the mail (or via delivery, etc.) prior to assaying the sample. In some such cases, the biological sample was “extracted” or “isolated” from an individual by another party prior to mailing (i.e., delivery, transfer, etc.), and then “obtained” by the testing facility upon arrival of the sample. Thus, a testing facility can obtain the sample and then assay the sample, thereby producing data related to the sample.

The terms “obtained” or “obtaining” as used herein can also include the physical extraction or isolation of a biological sample from a subject. Accordingly, a biological sample can be isolated from a subject (and thus “obtained”) by the same person or same entity that subsequently assays the sample. When a biological sample is “extracted” or “isolated” from a first party or entity and then transferred (e.g., delivered, mailed, etc.) to a second party, the sample was “obtained” by the first party (and also “isolated” by the first party), and then subsequently “obtained” (but not “isolated”) by the second party. Accordingly, in some embodiments, the step of obtaining does not comprise the step of isolating a biological sample.

In some embodiments, the step of obtaining comprises the step of isolating a biological sample (e.g., a pre-treatment biological sample, a post-treatment biological sample, etc.). Methods and protocols for isolating various biological samples (e.g., a blood sample, a serum sample, a plasma sample, a biopsy sample, an aspirate, etc.) will be known to one of ordinary skill in the art and any convenient method may be used to isolate a biological sample.

The terms “determining”, “measuring”, “evaluating”, “assessing,” “assaying,” and “analyzing” are used interchangeably herein to refer to any form of measurement, and include determining if an element is present or not. These terms include both quantitative and/or qualitative determinations. Assaying may be relative or absolute. For example, “assaying” can be determining whether the expression level is less than or “greater than or equal to” a particular threshold, (the threshold can be pre-determined or can be determined by assaying a control sample). On the other hand, “assaying to determine the expression level” can mean determining a quantitative value (using any convenient metric) that represents the level of expression (i.e., expression level, e.g., the amount of protein and/or RNA, e.g., mRNA).

Anti-CD47 agent. As used herein, the term “anti-CD47 agent” or “CD47-blocking agent” refers to any agent that reduces the binding of CD47 (e.g., on a target cell) to SIRPα (e.g., on a phagocytic cell). Non-limiting examples of suitable anti-CD47 reagents include SIRPα polypeptides, e.g. high affinity SIRPα polypeptides; anti-SIRPα antibodies; soluble CD47 polypeptides; and anti-CD47 antibodies or antibody fragments; and conjugates thereof, e.g. soluble SIRPα polypeptides conjugated to an Fc region polypeptide. In some embodiments, a suitable anti-CD47 agent specifically binds CD47 to reduce the binding of CD47 to SIRPα.

In some embodiments, a suitable anti-CD47 agent, e.g., an anti-SIRPα antibody, a soluble CD47 polypeptide, etc., specifically binds to SIRPα to reduce the binding of CD47 to SIRPα. A suitable anti-CD47 agent that binds SIRPα does not activate SIRPα (e.g., in the SIRPα-expressing phagocytic cell). The efficacy of a suitable anti-CD47 agent can be assessed by assaying the agent (further described below). In an exemplary assay, target cells are incubated in the presence or absence of the candidate agent. An agent for use in the methods of the invention will up-regulate phagocytosis by at least 5% (e.g., at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 500%, at least 1000%) compared to phagocytosis in the absence of the agent. Similarly, an in vitro assay for levels of tyrosine phosphorylation of SIRPα will show a decrease in phosphorylation by at least 5% (e.g., at least 10%, at least 15%, at least 20%, at least 30%, at least 40%, at least 50%, at least 60%, at least 70%, at least 80%, at least 90%, or 100%) compared to phosphorylation observed in absence of the candidate agent.

In some embodiments, the anti-CD47 agent does not activate CD47 upon binding. When CD47 is activated, a process akin to apoptosis (i.e., programmed cell death) may occur (Manna and Frazier (2004) Cancer Research, 64, 1026-1036). Thus, in some embodiments, the anti-CD47 agent does not directly induce cell death of a CD47-expressing cell.

SIRPα polypeptide. A SIRPα polypeptide comprises the portion of SIRPα that is sufficient to bind CD47 at a recognizable affinity, which portion normally lies between the signal sequence and the transmembrane domain, or a fragment thereof that retains the binding activity. A suitable SIRPα polypeptide reduces (e.g., blocks, prevents, etc.) the interaction between the native proteins SIRPα and CD47. The SIRPα reagent will usually comprise at least the dl domain of SIRPα. In some embodiments, a SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

Included as a SIRPα polypeptide are high-affinity variants of SIRPα as known and used in the art, including without limitation CV1-hIgG4, which has the set of amino acid substitutions relative to wild-type SIRPα of V61; V271; 131F; E47V; K53R; E54Q; H56P; S66T; V921 and is fused to an Fc region. High affinity SIRPα reagents are described in international application PCT/US13/21937, which is hereby specifically incorporated by reference. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more. The high affinity SIRPα reagent will usually comprise at least the dl domain of SIRPα with modified amino acid residues to increase affinity. The amino acid changes that provide for increased affinity are localized in the dl domain, and thus high affinity SIRPα reagents comprise a di domain of human SIRPα, with at least one amino acid change relative to the wild-type sequence within the di domain. Such a high affinity SIRPα reagent optionally comprises additional amino acid sequences, for example antibody Fc sequences; portions of the wild-type human SIRPα protein other than the dl domain, including without limitation residues 150 to 374 of the native protein or fragments thereof, usually fragments contiguous with the dl domain; and the like. High affinity SIRPα reagents may be monomeric or multimeric, i.e. dimer, trimer, tetramer, etc.

Anti-CD47 antibodies. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα (and are therefore not considered to be an “anti-CD47 agent” herein) and such an antibody can be referred to as a “non-blocking anti-CD47 antibody.” A suitable anti-CD47 antibody that is an “anti-CD47 agent” can be referred to as a “CD47-blocking antibody”. A non-limiting example of a non-blocking antibody is anti-CD47 antibody 2D3, which binds to CD47, but does not reduce the interaction between CD47 and SIRPα. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized or chimeric versions of such antibodies. Humanized antibodies (e.g., hu5F9-G4) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Anti-SIRPα antibodies. Antibodies that specifically bind to human SIRPα are known and used in the art, and may be adapted by the use of an engineered Fc region. Exemplary antibodies include those described in international patent application WO 2015/138600; in published US application 2014/0242095 (University Health Networks); published application CN103665165 (JIANGSU KUANGYA BIOLOGICAL MEDICAL SCIENCE & TECHNOLOGY; Zhao X W et al. Proc Natl Acad Sci USA 108:18342-7 (2011), each herein specifically incorporated by reference. An anti-SIRPα antibody may be pan-specific, i.e. binding to two or more different human SIRPα isoforms; or may be specific for one isoform. For example, the antibody 1.23A described by Zhang et al., supra. is reported to be specific for the SIRPα1 variant, while the 12C4 antibody is pan-specific. Anti-SIRPα antibodies can also be specific for SIRPα and lack binding to SIRPβ and/or SIRPγ. Anti-SIRPα antibodies can be pan-specific with respect to SIRPβ and/or SIRPγ.

Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Soluble CD47 polypeptides. In some embodiments, a subject anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis. In some cases, a suitable soluble CD47 polypeptide can be a fusion protein (for example as structurally described in US Patent Publication US20100239579, herein specifically incorporated by reference). However, only fusion proteins that do not activate/stimulate SIRPα are suitable for the methods provided herein. Suitable soluble CD47 polypeptides also include any peptide or peptide fragment comprising variant or naturally existing CD47 sequences (e.g., extracellular domain sequences or extracellular domain variants) that can specifically bind SIRPα and inhibit the interaction between CD47 and SIRPα without stimulating enough SIRPα activity to inhibit phagocytosis.

In certain embodiments, soluble CD47 polypeptide comprises the extracellular domain of CD47, including the signal peptide, such that the extracellular portion of CD47 is typically 142 amino acids in length, and has the amino acid sequence set forth in, for example, the Genbank reference sequence for human CD47, including NP_942088 or NP_001768.1. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

In certain embodiments, the signal peptide amino acid sequence may be substituted with a signal peptide amino acid sequence that is derived from another polypeptide (e.g., for example, an immunoglobulin or CTLA4). For example, unlike full-length CD47, which is a cell surface polypeptide that traverses the outer cell membrane, the soluble CD47 polypeptides are secreted; accordingly, a polynucleotide encoding a soluble CD47 polypeptide may include a nucleotide sequence encoding a signal peptide that is associated with a polypeptide that is normally secreted from a cell.

In other embodiments, the soluble CD47 polypeptide comprises an extracellular domain of CD47 that lacks the signal peptide (124 amino acids). As described herein, signal peptides are not exposed on the cell surface of a secreted or transmembrane protein because either the signal peptide is cleaved during translocation of the protein or the signal peptide remains anchored in the outer cell membrane (such a peptide is also called a signal anchor). The signal peptide sequence of CD47 is believed to be cleaved from the precursor CD47 polypeptide in vivo.

In other embodiments, a soluble CD47 polypeptide comprises a CD47 extracellular domain variant. Such a soluble CD47 polypeptide retains the capability to bind to SIRPα without stimulating SIRPα signaling. The CD47 extracellular domain variant may have an amino acid sequence that is at least 65%-75%, 75%-80%, 80-85%, 85%-90%, or 95%-99% identical (which includes any percent identity between any one of the described ranges) to a reference human CD47 sequence.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom but has not yet been diagnosed as having it; (b) inhibiting the disease and/or symptom(s), i.e., arresting their development; or (c) relieving the disease symptom(s), i.e., causing regression of the disease and/or symptom(s). Those in need of treatment include those already inflicted (e.g., those with cancer, those with an infection, etc.) as well as those in which prevention is desired (e.g., those with increased susceptibility to cancer, those suspected of having cancer, etc.).

A target cell can have cancer, can harbor an infection (e.g., a chronic infection), and other hyper-proliferative conditions, for example sclerosis, fibrosis, and the like, etc. “Inflicted cells” may be those cells that cause the symptoms, illness, or disease. As non-limiting examples, the inflicted cells of a patient can be cancer cells, infected cells, and the like.

A therapeutic treatment is one in which the subject is inflicted prior to administration and a prophylactic treatment is one in which the subject is not inflicted prior to administration. In some embodiments, the subject has an increased likelihood of becoming inflicted or is suspected of being inflicted prior to treatment. In some embodiments, the subject is suspected of having an increased likelihood of becoming inflicted.

Examples of symptoms, illnesses, and/or diseases that can be treated with an anti-CD47 agent include, but are not limited to cancer and infection (e.g., chronic infection). As used herein “cancer” includes any form of cancer (e.g., leukemia; acute myeloid leukemia (AML); acute lymphoblastic leukemia (ALL); metastasis; minimal residual disease; solid tumor cancers, e.g., lung, prostate, breast, bladder, colon, ovarian, glioblastoma, medulloblastoma, leiomyosarcoma, and head & neck squamous cell carcinomas, melanomas; etc.). Any cancer, where the cancer cells exhibit increased expression of CD47 or pro-phagocytic “eat me” signals compared to non-cancer cells, is a suitable cancer to be treated by the subject methods and kits.

As used herein, the term “infection” refers to any state in at least one cell of an organism (i.e., a subject) is infected by an infectious agent (e.g., a subject has an intracellular pathogen infection, e.g., a chronic intracellular pathogen infection). As used herein, the term “infectious agent” refers to a foreign biological entity (i.e. a pathogen) that induces increased CD47 expression or upregulation of pro-phagocytic “eat me” signals in at least one cell of the infected organism. For example, infectious agents include, but are not limited to bacteria, viruses, protozoans, and fungi. Intracellular pathogens are of particular interest. Infectious diseases are disorders caused by infectious agents. Some infectious agents cause no recognizable symptoms or disease under certain conditions, but have the potential to cause symptoms or disease under changed conditions. The subject methods can be used in the treatment of chronic pathogen infections, for example including but not limited to viral infections, e.g. retrovirus, lentivirus, hepadna virus, herpes viruses, pox viruses, human papilloma viruses, etc.; intracellular bacterial infections, e.g. Mycobacterium, Chlamydophila, Ehrlichia, Rickettsia, Brucella, Legionella, Francisella, Listeria, Coxiella, Neisseria, Salmonella, Yersinia sp, Helicobacter pylori etc.; and intracellular protozoan pathogens, e.g. Plasmodium sp, Trypanosoma sp., Giardia sp., Toxoplasma sp., Leishmania sp., etc.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

A “therapeutically effective dose” or “therapeutic dose” is an amount sufficient to effect desired clinical results (i.e., achieve therapeutic efficacy). A therapeutically effective dose can be administered in one or more administrations. For purposes of this invention, a therapeutically effective dose of an anti-CD47 agent is an amount that is sufficient to palliate, ameliorate, stabilize, reverse, prevent, slow or delay the progression of the disease state (e.g., cancer or chronic infection) by increasing phagocytosis of a target cell (e.g., a target cell). Thus, a therapeutically effective dose of an anti-CD47 agent reduces the binding of CD47 on a target cell, to SIRPα on a phagocytic cell, at an effective dose for increasing the phagocytosis of the target cell.

In some embodiments, a therapeutically effective dose is one that provides for sustained serum levels of anti-CD47 agent (e.g., an anti-CD47 antibody) of about 40 μg/ml or more (e.g, about 50 μg/ml or more, about 60 μg/ml or more, about 75 μg/ml or more, about 100 μg/ml or more, about 125 μg/ml or more, or about 150 μg/ml or more). In some embodiments, a therapeutically effective dose leads to sustained serum levels of anti-CD47 agent (e.g., an anti-CD47 antibody) that range from about 40 μg/ml to about 300 μg/ml (e.g, from about 40 μg/ml to about 250 μg/ml, from about 40 μg/ml to about 200 μg/ml, from about 40 μg/ml to about 150 μg/ml, from about 40 μg/ml to about 100 μg/ml, from about 50 μg/ml to about 300 μg/ml, from about 50 μg/ml to about 250 μg/ml, from about 50 μg/ml to about 200 μg/ml, from about 50 μg/ml to about 150 μg/ml, from about 75 μg/ml to about 300 μg/ml from about 75 μg/ml to about 250 μg/ml, from about 75 μg/ml to about 200 μg/ml, from about 75 μg/ml to about 150 μg/ml, from about 100 μg/ml to about 300 μg/ml, from about 100 μg/ml to about 250 μg/ml, or from about 100 μg/ml to about 200 μg/ml). In some embodiments, a therapeutically effective dose for treating solid tumors provides for sustained serum levels of anti-CD47 agent (e.g., an anti-CD47 antibody) of about 100 μg/ml or more (e.g., sustained serum levels that range from about 100 μg/ml to about 200 μg/ml). In some embodiments, a therapeutically effective dose for treating non-solid tumors (e.g., acute myeloid leukemia (AML)) provides for sustained serum levels of anti-CD47 agent (e.g., an anti-CD47 antibody) of about 50 μg/ml or more (e.g., sustained serum levels of 75 μg/ml or more; or sustained serum levels that range from about 50 μg/ml to about 150 μg/ml).

Accordingly, a single therapeutically effective dose or a series of therapeutically effective doses would be able to achieve and maintain a serum level of anti-CD47 agent. A therapeutically effective dose of an anti-CD47 agent can depend on the specific agent used, but is usually about 2 mg/kg body weight or more (e.g., about 2 mg/kg or more, about 4 mg/kg or more, about 8 mg/kg or more, about 10 mg/kg or more, about 15 mg/kg or more, about 20 mg/kg or more, about 25 mg/kg or more, about 30 mg/kg or more, about 35 mg/kg or more, or about 40 mg/kg or more), or from about 10 mg/kg to about 40 mg/kg (e.g., from about 10 mg/kg to about 35 mg/kg, or from about 10 mg/kg to about 30 mg/kg). The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art.

A sub-therapeutic dose is a dose (i.e., an amount) that is not sufficient to effect the desired clinical results. For example, a sub-therapeutic dose of an anti-CD47 agent is an amount that is not sufficient to palliate, ameliorate, stabilize, reverse, prevent, slow or delay the progression of the disease state (e.g., cancer, infection, inflammation, etc.). In some cases, it is desirable to use a sub-therapeutic dose of an anti-CD47 agent as a primer agent (described in more detail below). While the use of a sub-therapeutic dose of an anti-CD47 agent as a primer agent achieves a desired outcome (e.g., the subject is “primed” to receive a therapeutically effective dose), the dose is not considered to be a “therapeutic dose” because the sub-therapeutic dose does not effectively increase phagocytosis of a target cell and is not sufficient to palliate, ameliorate, stabilize, reverse, prevent, slow or delay the progression of the disease state. A sub-therapeutic dose of an anti-CD47 agent can depend on the specific agent used, but is generally less than about 10 mg/kg.

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides, or binding of a SIRPα polypeptide). In some embodiments, the affinity of one molecule for another molecule to which it specifically binds is characterized by a K_{D} (dissociation constant) of 10⁻⁵M or less (e.g., 10⁻⁶M or less, 10⁻⁷M or less, 10⁻⁸M or less, 10⁻⁹M or less, 10⁻¹⁰M or less, 10⁻¹¹M or less, 10⁻¹²M or less, 10⁻¹³M or less, 10⁻¹⁴M or less, 10⁻¹⁵M or less, or 10⁻¹⁸M or less). “Affinity” refers to the strength of binding, increased binding affinity being correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member). Suitable specific binding members include agents that specifically bind CD47 and/or SIRPα (i.e., anti-CD47 agents), or that otherwise block the interaction between CD47 and SIRPα.

The terms “phagocytic cells” and “phagocytes” are used interchangeably herein to refer to a cell that is capable of phagocytosis. There are three main categories of phagocytes: macrophages and mononuclear cells (histiocytes and monocytes); polymorphonuclear leukocytes (neutrophils); and dendritic cells.

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibodies” (Abs) and “immunoglobulins” (Igs) are glycoproteins having the same structural characteristics. While antibodies exhibit binding specificity to a specific antigen, immunoglobulins include both antibodies and other antibody-like molecules which lack antigen specificity. Polypeptides of the latter kind are, for example, produced at low levels by the lymph system and at increased levels by myelomas.

“Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety and (4) nanobodies comprising single Ig domains from non-human species or other specific single-domain binding modules; and multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this invention, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

“Providing an analysis” is used herein to refer to the delivery of an oral or written analysis (i.e., a document, a report, etc.). A written analysis can be a printed or electronic document. A suitable analysis (e.g., an oral or written report) provides any or all of the following information: identifying information of the subject (name, age, etc.), a description of what type of biological sample(s) was used and/or how it was used, the technique used to assay the sample, the results of the assay, the assessment as to whether the individual is determined to be responsive or not responsive to the anti-CD47 agent, a recommendation to continue or after therapy, a recommended strategy for additional therapy, etc. The report can be in any format including, but not limited to printed information on a suitable medium or substrate (e.g., paper); or electronic format. If in electronic format, the report can be in any computer readable medium, e.g., diskette, compact disk (CD), flash drive, and the like, on which the information has been recorded. In addition, the report may be present as a website address which may be used via the internet to access the information at a remote site.

### Methods

Methods are provided for determining whether an individual is responsive to an anti-CD47 agent. It has been found that individuals vary in basal and induced levels of SIRPα expression on immune cells, e.g. dendritic cells, monocytes, macrophages and CAR T cells, etc. It is further shown that differences in SIRPα expression are associated with responsiveness to therapeutic CD47 blockade, where cells that express higher SIRPα levels are less responsive to the blockade. Genotypic analysis has identified SNP polymorphisms that predict SIRPα expression levels, which analysis is useful in identifying individuals for treatment.

A DNA sample from an individual is assayed to determine the genotype with respect to polymorphisms that affect expression of SIRPα. In some embodiments the polymorphism is linked to the SNP rs4813322. In some embodiments the polymorphism is SNP rs4813322. An individual may be genotyped as homozygous A/A; homozygous G/G or heterozygous A/G at SNP rs4813322. Presence of one or two A alleles at SNP rs4813322 indicates higher SIRPα expression, and decreased responsiveness to CD47 blockade, where homozygous AA individuals are indicated to have highest levels of SIRPα expression. Genotyping results are optionally confirmed with a direct measurement of SIRPα expression levels in a relevant cell population, e.g. monocytes, macrophages, dendritic cells, etc.

In other embodiments an SNP is selected from those listed in Table 1.

Alternatively, a sample from an individual comprising cells that express SIRPα, including for example monocytes, macrophages, dendritic cells, etc., can be analyzed for basal levels of SIRPα expression, where cells expressing high levels, relative to a normal control, are indicated as less responsive to therapeutic CD47 blockade.

In some embodiments, an individual determined to be of a responsive type is treated with an anti-CD47 agent, i.e. by therapeutic CD47 blockade. In some embodiments, the methods of the invention find use in determining whether to continue or after therapy, where an individual responsive to CD47 blockade may be treated with the same. In some such embodiments, the individual is being treated with an anti-CD47 agent for cancer. In other embodiments the individual is being treated with an anti-CD47 agent for infection, particularly with an intracellular pathogen.

### Genotyping Methods

Individuals may be genotyped by any convenient method in the art for detecting polymorphisms, for example in rs4813322 or polymorphisms linked to rs4813322 or in other SNP associated with SIRPα expression. For example, where a subject is genotyped for a single nucleotide polymorphism (SNP), a subject or patient sample, e.g., cells or collections thereof, are assayed to determine the nucleotide sequence of the gene at that polymorphism by using one or more genotyping reagents, such as but not limited to sequencing-, hybridization-, amplification-reagents, including primers, etc., which may or may not be labeled, as described below, amplification or sequencing enzymes, buffers, etc.

A genetic sample obtained from the subject is assayed to determine the genotype of the subject with respect to at least one, i.e., one or more, polymorphisms. Any convenient protocol for assaying a sample for the above one or more target polymorphisms may be employed. The target polymorphism is generally detected at the nucleic acid level, e.g., by assaying for the presence of nucleic acid polymorphism, e.g., a single nucleotide polymorphism (SNP) that affects expression of the SIRPα protein.

Any biological sample that comprises a polynucleotide from the individual is suitable for use in the methods of the invention. The biological sample may be processed so as to isolate the polynucleotide. Alternatively, whole cells or other biological samples may be used without isolation of the polynucleotides contained therein. Detection of a target polymorphism in a polynucleotide sample derived from an individual can be accomplished by any means known in the art, including, but not limited to, amplification of a sequence with specific primers; determination of the nucleotide sequence of the polynucleotide sample; hybridization analysis; single strand conformational polymorphism analysis; denaturing gradient gel electrophoresis; mismatch cleavage detection; and the like. Detection of a target polymorphism can also be accomplished by detecting an alteration in the level of a mRNA transcript of the gene; aberrant modification of the corresponding gene, e.g., an aberrant methylation pattern; an alteration in the level of the corresponding polypeptide; and/or an alteration in corresponding polypeptide activity. An advantage of genotyping is the ability to conduct the analysis using any cell sample, rather than requiring a sample of SIRPα-expressing cells.

Detection of a target polymorphism by analyzing a polynucleotide sample can be conducted in a number of ways. A test nucleic acid sample can be amplified with primers which amplify a region known to comprise the target polymorphism(s). Genomic DNA or mRNA can be used directly. Alternatively, the region of interest can be cloned into a suitable vector and grown in sufficient quantity for analysis. The nucleic acid may be amplified by conventional techniques, such as a polymerase chain reaction (PCR), to provide sufficient amounts for analysis. The use of the polymerase chain reaction is described in a variety of publications, including, e.g., “PCR Protocols (Methods in Molecular Biology)” (2000) J. M. S. Bartlett and D. Stirling, eds, Humana Press; and “PCR Applications: Protocols for Functional Genomics” (1999) Innis, Gelfand, and Sninsky, eds., Academic Press. Once the region comprising a target polymorphism has been amplified, the target polymorphism can be detected in the PCR product by nucleotide sequencing, by SSCP analysis, or any other method known in the art.

PCR may also be used to determine whether a polymorphism is present by using a primer that is specific for the polymorphism. Such methods may comprise the steps of collecting from an individual a biological sample comprising the individual's genetic material as template, optionally isolating template nucleic acid (genomic DNA, mRNA, or both) from the biological sample, contacting the template nucleic acid sample with one or more primers that specifically hybridize with a target polymorphic nucleic acid molecule under conditions such that hybridization and amplification of the template nucleic acid molecules in the sample occurs, and detecting the presence, absence, and/or relative amount of an amplification product and comparing the length to a control sample. Observation of an amplification product of the expected size is an indication that the target polymorphism contained within the target polymorphic primer is present in the test nucleic acid sample. Parameters such as hybridization conditions, polymorphic primer length, and position of the polymorphism within the polymorphic primer may be chosen such that hybridization will not occur unless a polymorphism present in the primer(s) is also present in the sample nucleic acid. Those of ordinary skill in the art are well aware of how to select and vary such parameters. See, e.g., Saiki et al. (1986) Nature 324:163; and Saiki et al (1989) Proc. Natl. Acad. Sci. USA 86:6230.

Alternatively, various methods are known in the art that utilize oligonucleotide ligation as a means of detecting polymorphisms. See, e.g., Riley et al. (1990) Nucleic Acids Res. 18:2887-2890; and Delahunty et al. (1996) Am. J. Hum. Genet. 58:1239-1246.

A detectable label may be included in an amplification reaction. Suitable labels include fluorochromes, e.g. fluorescein isothiocyanate (FITC), rhodamine, Texas Red, phycoerythrin, allophycocyanin, 6 carboxyfluorescein (6 FAM), 2′,7′ dimethoxy 4′,5′ dichloro 6 carboxyfluorescein (JOE), 6 carboxy X rhodamine (ROX), 6 carboxy 2′,4′,7′,4,7 hexachlorofluorescein (HEX), 5 carboxyfluorescein (5 FAM) or N,N,N′,N′ tetramethyl 6 carboxyrhodamine (TAMRA), radioactive labels, e.g. 32P, 35S, 3H; etc. The label may be a two stage system, where the amplified DNA is conjugated to biotin, haptens, etc. having a high affinity binding partner, e.g. avidin, specific antibodies, etc., where the binding partner is conjugated to a detectable label. The label may be conjugated to one or both of the primers. Alternatively, the pool of nucleotides used in the amplification is labeled, so as to incorporate the label into the amplification product.

The sample nucleic acid may be sequenced using any convenient sequencing protocol. Sequencing platforms that can be used in the present disclosure include but are not limited to: pyrosequencing, sequencing-by-synthesis, single-molecule sequencing, second-generation sequencing, nanopore sequencing, sequencing by ligation, or sequencing by hybridization. Preferred sequencing platforms are those commercially available from Illumina (RNA-Seq) and Helicos (Digital Gene Expression or “DGE”). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

Genomic DNA or mRNA may be used directly. If mRNA is used, a cDNA copy may first be made. If desired, the sample nucleic acid can be amplified using a PCR. A variety of sequencing reactions known in the art can be used to directly sequence the relevant gene, or a portion thereof in which a specific polymorphism is known to occur, and detect polymorphisms by comparing the sequence of the sample nucleic acid with a reference polynucleotide that contains a target polymorphism.

Hybridization with the variant sequence may also be used to determine the presence of a target polymorphism. Hybridization analysis can be carried out in a number of different ways, including, but not limited to Southern blots, Northern blots, dot blots, microarrays, etc. The hybridization pattern of a control and variant sequence to an array of oligonucleotide probes immobilized on a solid support, as described in U.S. Pat. No. 5,445,934, or in WO 95/35505, may also be used protocols for detecting the presence of variant sequences. Identification of a polymorphism in a nucleic acid sample can be performed by hybridizing a sample and control nucleic acids to high density arrays containing hundreds or thousands of oligonucleotide probes. See, e.g. Cronin et al. (1996) Human Mutation 7:244-255; and Kozal et al. (1996) Nature Med. 2:753-759.

Single strand conformational polymorphism (SSCP) analysis; denaturing gradient gel electrophoresis (DGGE); mismatch cleavage detection; and heteroduplex analysis in gel matrices can also be used to detect polymorphisms. Alternatively, where a polymorphism creates or destroys a recognition site for a restriction endonuclease (restriction fragment length polymorphism, RFLP), the sample is digested with that endonuclease, and the products size fractionated to determine whether the fragment was digested. Fractionation is performed by gel or capillary electrophoresis, particularly acrylamide or agarose gels. The aforementioned techniques are well known in the art. Detailed description of these techniques can be found in a variety of publications, including, e.g., “Laboratory Methods for the Detection of Mutations and Polymorphisms in DNA” (1997) G. R. Taylor, ed., CRC Press, and references cited therein.

It will be understood by one of ordinary skill in the art that in some cases, it is convenient to wait until multiple samples (e.g., a pre-treatment biological sample and a post-treatment biological sample) have been obtained prior to assaying the samples. Accordingly, in some cases an isolated biological sample (e.g., a pre-treatment biological sample, a post-treatment biological sample, etc.) is stored until all appropriate samples have been obtained. One of ordinary skill in the art will understand how to appropriately store a variety of different types of biological samples and any convenient method of storage may be used (e.g., refrigeration) that is appropriate for the particular biological sample. In some embodiments, a pre-treatment biological sample is assayed prior to obtaining a post-treatment biological sample. In some cases, a pre-treatment biological sample and a post-treatment biological sample are assayed in parallel. In some cases, multiple different post-treatment biological samples and/or a pre-treatment biological sample are assayed in parallel. In some cases, biological samples are processed immediately or as soon as possible after they are obtained.

### Expression Detection

In some methods, the concentration (i.e., “level”), or expression level of SIRPα protein, etc., in a biological sample is measured (i.e., “determined”). By “expression level” (or “level”) it is meant the level of gene product (e.g. the absolute and/or normalized value determined for the RNA expression level of SIRPα or for the expression level of the encoded polypeptide, or the concentration of the protein on a relevant cell). The term “gene product” or “expression product” are used herein to refer to the RNA transcription products of the gene, including mRNA, and the polypeptide translation products of such RNA transcripts.

The level of expression can be expressed in arbitrary units associated with a particular assay (e.g., fluorescence units, e.g., mean fluorescence intensity (MFI)), or can be expressed as an absolute value with defined units (e.g., number of mRNA transcripts, number of protein molecules, concentration of protein, etc.). Additionally, the level of expression of SIRPα can be compared to the expression level of one or more additional genes (e.g., nucleic acids and/or their encoded proteins) to derive a normalized value that represents a normalized expression level. The specific metric (or units) chosen is not crucial as long as the same units are used (or conversion to the same units is performed) when evaluating multiple biological samples from the same individual (e.g., biological samples taken at different points in time from the same individual). This is because the units cancel when calculating a fold-change (i.e., determining a ratio) in the expression level from one biological sample to the next (e.g., biological samples taken at different points in time from the same individual).

For measuring RNA levels, the amount or level of an RNA in the sample is determined. In some instances, the expression level of one or more additional RNAs may also be measured, and the level of SIRPα expression compared to the level of the one or more additional RNAs to provide a normalized value for the SIRPα expression level. Any convenient protocol for evaluating RNA levels may be employed wherein the level of one or more RNAs in the assayed sample is determined.

A number of exemplary methods for measuring RNA (e.g., mRNA) expression levels (e.g., expression level of a SIRPα nucleic acid) in a sample are known by one of ordinary skill in the art, and any convenient method can be used. Exemplary methods include, but are not limited to: hybridization-based methods (e.g., Northern blotting, array hybridization (e.g., microarray); in situ hybridization; in situ hybridization followed by FACS; and the like)(Parker & Barnes, Methods in Molecular Biology 106:247-283 (1999)); RNAse protection assays (Hod, Biotechniques 13:852-854 (1992)); PCR-based methods (e.g., reverse transcription PCR (RT-PCR), quantitative RT-PCR (qRT-PCR), real-time RT-PCR, etc.)(Weis et al., Trends in Genetics 8:263-264 (1992)); nucleic acid sequencing methods (e.g., Sanger sequencing, Next Generation sequencing (i.e., massive parallel high throughput sequencing, e.g., Illumina's reversible terminator method, Roche's pyrosequencing method (454), Life Technologies' sequencing by ligation (the SOLiD platform), Life Technologies' Ion Torrent platform, single molecule sequencing, etc.); and the like.

For measuring mRNA levels, the starting material is typically total RNA or poly A+ RNA isolated from a biological sample (e.g., suspension of cells from a peripheral blood sample, a bone marrow sample, etc., or from a homogenized tissue, e.g. a homogenized biopsy sample, an aspirate, a homogenized paraffin- or OCT-embedded sample, etc.). General methods for mRNA extraction are well known in the art and are disclosed in standard textbooks of molecular biology, including Ausubel et al., Current Protocols of Molecular Biology, John Wiley and Sons (1997). RNA isolation can also be performed using a purification kit, buffer set and protease from commercial manufacturers, according to the manufacturers instructions. For example, RNA from cell suspensions can be isolated using Qiagen RNeasy mini-columns, and RNA from cell suspensions or homogenized tissue samples can be isolated using the TRIzol reagent-based kits (Invitrogen), MasterPure™ Complete DNA and RNA Purification Kit (EPICENTRE™, Madison, Wis.), Paraffin Block RNA Isolation Kit (Ambion, Inc.) or RNA Stat-60 kit (Tel-Test).

A variety of different manners of measuring mRNA levels are known in the art, e.g. as employed in the field of differential gene expression analysis. One representative and convenient type of protocol for measuring mRNA levels is array-based gene expression profiling. Such protocols are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively.

Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the phenotype determinative genes whose expression is being assayed is contacted with target nucleic acids as described above. Contact is carried out under hybridization conditions, e.g., stringent hybridization conditions, and unbound nucleic acid is then removed. The term “stringent assay conditions” as used herein refers to conditions that are compatible to produce binding pairs of nucleic acids, e.g., surface bound and solution phase nucleic acids, of sufficient complementarity to provide for the desired level of specificity in the assay while being less compatible to the formation of binding pairs between binding members of insufficient complementarity to provide for the desired specificity. Stringent assay conditions are the summation or combination (totality) of both hybridization and wash conditions.

The resultant pattern of hybridized nucleic acid provides information regarding expression for each of the genes that have been probed, where the expression information is in terms of whether or not the gene is expressed and, typically, at what level, where the expression data, i.e., expression profile (e.g., in the form of a transcriptosome), may be both qualitative and quantitative.

Alternatively, non-array based methods for quantitating the level of one or more nucleic acids in a sample may be employed. These include those based on amplification protocols, e.g., Polymerase Chain Reaction (PCR)-based assays, including quantitative PCR, reverse-transcription PCR (RT-PCR), real-time PCR, and the like, e.g. TaqMan4 RT-PCR, MassARRAY® System, BeadArray® technology, and Luminex technology; and those that rely upon hybridization of probes to filters, e.g. Northern blotting and in situ hybridization.

For measuring protein levels, the amount or level of a polypeptide in the biological sample is determined. In some embodiments concentration is a relative value measured by comparing the level of one protein relative to another protein. In other embodiments the concentration is an absolute measurement of weight/volume or weight/weight.

In some cases, the cells are removed from the biological sample (e.g., via centrifugation, via adhering cells to a dish or to plastic, etc.) prior to measuring the concentration. In some cases, the intracellular protein level is measured by lysing the removed cells of the biological sample to measure the level of protein in the cellular contents. In some cases, both the extracellular and intracellular levels of protein are measured by separating the cellular and fluid portions of the biological sample (e.g., via centrifugation), measuring the extracellular level of the protein by measuring the level of protein in the fluid portion of the biological sample, and measuring the intracellular level of protein by measuring the level of protein in the cellular portion of the biological sample (e.g., after lysing the cells). In some cases, the total level of protein (i.e., combined extracellular and intracellular protein) is measured by lysing the cells of the biological sample to include the intracellular contents as part of the sample.

In some instances, the concentration of one or more additional proteins may also be measured, and SIRPα concentration compared to the level of the one or more additional proteins to provide a normalized value for the SIRPα concentration. Any convenient protocol for evaluating protein levels may be employed wherein the level of one or more proteins in the assayed sample is determined.

While a variety of different manners of assaying for protein levels are known to one of ordinary skill in the art and any convenient method may be used, one representative and convenient type of protocol for assaying protein levels is ELISA, an antibody-based method. In ELISA and ELISA-based assays, one or more antibodies specific for the proteins of interest may be immobilized onto a selected solid surface, preferably a surface exhibiting a protein affinity such as the wells of a polystyrene microtiter plate. After washing to remove incompletely adsorbed material, the assay plate wells are coated with a non-specific “blocking” protein that is known to be antigenically neutral with regard to the test sample such as bovine serum albumin (BSA), casein or solutions of powdered milk. This allows for blocking of non-specific adsorption sites on the immobilizing surface, thereby reducing the background caused by non-specific binding of antigen onto the surface. After washing to remove unbound blocking protein, the immobilizing surface is contacted with the sample to be tested under conditions that are conducive to immune complex (antigen/antibody) formation. Following incubation, the antisera-contacted surface is washed so as to remove non-immunocomplexed material. The occurrence and amount of immunocomplex formation may then be determined by subjecting the bound immunocomplexes to a second antibody having specificity for the target that differs from the first antibody and detecting binding of the second antibody. In certain embodiments, the second antibody will have an associated enzyme, e.g. urease, peroxidase, or alkaline phosphatase, which will generate a color precipitate upon incubating with an appropriate chromogenic substrate. After such incubation with the second antibody and washing to remove unbound material, the amount of label is quantified, for example by incubation with a chromogenic substrate such as urea and bromocresol purple in the case of a urease label or 2,2′-azino-di-(3-ethyl-benzthiazoline)-6-sulfonic acid (ABTS) and H2O2, in the case of a peroxidase label. Quantitation is then achieved by measuring the degree of color generation, e.g., using a visible spectrum spectrophotometer.

The preceding format may be altered by first binding the sample to the assay plate. Then, primary antibody is incubated with the assay plate, followed by detecting of bound primary antibody using a labeled second antibody with specificity for the primary antibody. The solid substrate upon which the antibody or antibodies are immobilized can be made of a wide variety of materials and in a wide variety of shapes, e.g., microtiter plate, microbead, dipstick, resin particle, etc. The substrate may be chosen to maximize signal to noise ratios, to minimize background binding, as well as for ease of separation and cost. Washes may be effected in a manner most appropriate for the substrate being used, for example, by removing a bead or dipstick from a reservoir, emptying or diluting a reservoir such as a microtiter plate well, or rinsing a bead, particle, chromatographic column or filter with a wash solution or solvent.

Alternatively, non-ELISA based-methods for measuring the levels of one or more proteins in a sample may be employed. Representative exemplary methods include but are not limited to antibody-based methods (e.g., Western blotting, proteomic arrays, xMAP™ microsphere technology (e.g., Luminex technology), immunohistochemistry, flow cytometry, and the like) as well as non antibody-based methods (e.g., mass spectrometry).

The term “responsive” as used herein means that the anti-CD47 agent is having the desired effect and the individual's body is responding appropriately to the administration of the anti-CD47 agent. For example, and not to bound by theory, the administration of an anti-CD47 agent is expected to block the interaction between CD47 on a target cell and SIRPα on a phagocytic cell (e.g., macrophage). When this blockage is successful, the body responds in multiple ways, one of which includes the activation of phagocytic cells, which (i) no longer receive “don't eat me” signals from the target cell, (ii) begin to actively phagocytose the target cell.

The determination that an individual will be responsive to an anti-CD47 agent is a direct and active clinical application of the correlation between SIRPα expression and the activity of an anti-CD47 agent, where SIRPα expression can be directly measured, or can be inferred from genotyping as disclosed herein. For example, “determining” requires the active step of reviewing the data, which is produced during the active assaying step(s), and resolving whether an individual is or is not responsive (or maintaining responsiveness). Additionally, in some cases, a decision is made to proceed with the current treatment (i.e., therapy), or instead to alter the treatment. In some cases, the subject methods include the step of continuing therapy or altering therapy.

The term “continue treatment” (i.e., continue therapy) is used herein to mean that the current course of treatment (e.g., continued administration of an anti-CD47 agent) is to continue. For example, if the current course of treatment includes the administration of an anti-CD47 agent at a particular dose and/or with a particular dosing frequency (e.g., once per day, once every other day, etc.), than to “continue therapy” would be to continue administering the anti-CD47 agent at that particular dose and/or with that particular dosing frequency. If the current course of treatment includes a ramping (e.g., decreasing dose and/or frequency over time) of administration of an anti-CD47 agent, then “continue therapy” would mean to continue the ramping (e.g., until the individual is deemed to be non-responsive, at which point the therapy may be altered, e.g., the altered therapy may include an increased dose and/or frequency of an anti-CD47 agent).

Alternatively, “altering therapy” is used herein to mean “discontinuing therapy” or “changing the therapy” (e.g., changing the particular dose and/or frequency of anti-CD47 agent administration, e.g., increasing the dose and/or frequency). In some cases, therapy can be altered, e.g., increased, until a dose and/or frequency is reached at which the individual is deemed to be responsive. In some embodiments, altering therapy means changing which anti-CD47 agent is administered, discontinuing use of any anti-CD47 agent altogether, etc.

In some such cases, an anti-CD47 agent is administered to the individual more than once (e.g., two or more times, three or more times, four or more times, five or more times, etc.). When administered more than once, an anti-CD47 agent can be administered at the same dose or at a different does than previously administered.

The anti-CD47 agent can be administered to an individual any time after a pre-treatment biological sample is isolated from the individual. The anti-CD47 agent may be administered simultaneous with or as soon as possible (e.g., about 7 days or less, about 3 days or less, e.g., 2 days or less, 36 hours or less, 1 day or less, 20 hours or less, 18 hours or less, 12 hours or less, 9 hours or less, 6 hours or less, 3 hours or less, 2.5 hours or less, 2 hours or less, 1.5 hours or less, 1 hour or less, 45 minutes or less, 30 minutes or less, 20 minutes or less, 15 minutes or less, 10 minutes or less, 5 minutes or less, 2 minutes or less, or 1 minute or less) after a pre-treatment biological sample is isolated (or, when multiple pre-treatment biological samples are isolated, after the final pre-treatment biological sample is isolated).

In some cases, a second anti-CD47 agent is administered to the individual. The second anti-CD47 agent can be the same agent and/or same dose as a previously administered anti-CD47 agent. In some cases, the anti-CD47 agent is a different agent and/or a different dose than a previously administered anti-CD47 agent. Any anti-CD47 agent can be administered one or more times as described above and any post-treatment biological sample can be isolated from the individual after any administration of an anti-CD47 agent. Thus, for example, after a first post-treatment biological sample is collected, an anti-CD47 agent can be administered to the individual one or more times and another post-treatment biological sample (e.g., a second, third, fourth, fifth, etc. post-treatment biological sample) can be isolated after any of the administrations of the anti-CD47 agent. When an anti-CD47 agent is administered to an individual more than once or when more than one anti-CD47 agent is administered, each administration of an anti-CD47 agent can take place in a range from about 2 hours to about 8 weeks (e.g., about 2 hours to about 48 hours, about 2 hours to about 36 hours, about 2 hours to about 24 hours, about 2 hours to about 12 hours, about 2 hours to about 6 hours, about 12 hours to about 4 weeks, about 12 hours to about 2 weeks, about 12 hours to about 1 week, about 12 hours to about 2 days, about 12 hours to about 36 hours, about 1 day to about 8 weeks, about 1 day to about 6 weeks, about 1 day to about 4 weeks, about 1 day to about 2 weeks, about 1 day to about 1 week, about 3 days to about 8 weeks, about 3 days to about 6 weeks, about 3 days to about 4 weeks, about 3 days to about 2 weeks, about 3 days to about 1 week, about 1 week to about 8 weeks, about 1 week to about 6 weeks, or about 1 week to about 4 weeks) after a previous administration of an anti-CD47 agent.

In some embodiments, the subject methods include providing an analysis indicating whether the individual is determined to be responsive or not responsive to therapeutic CD47 blockade. As described above, an analysis can be an oral or written report (e.g., written or electronic document). The analysis can be provided to the subject, to the subject's physician, to a testing facility, etc. The analysis can also be accessible as a website address via the internet. In some such cases, the analysis can be accessible by multiple different entities (e.g., the subject, the subject's physician, a testing facility, etc.)

Administering an anti-CD47 agent. Suitable anti-CD47 agents can be provided in pharmaceutical compositions suitable for therapeutic use, e.g. for human treatment. In some embodiments, pharmaceutical compositions of the present invention include one or more therapeutic entities of the present invention or pharmaceutically acceptable salts, esters or solvates thereof. In some other embodiments, the use of an anti-CD47 agent includes use in combination with another therapeutic agent (e.g., another anti-infection agent or another anti-cancer agent). Therapeutic formulations comprising one or more anti-CD47 agents of the invention are prepared for storage by mixing the anti-CD47 agent having the desired degree of purity with optional physiologically acceptable carriers, excipients or stabilizers (Remington's Pharmaceutical Sciences 16th edition, Osol, A. Ed. (1980)), in the form of lyophilized formulations or aqueous solutions. The anti-CD47 agent composition will be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

The anti-CD47 agent can be “administered” by any suitable means, including topical, oral, parenteral, intrapulmonary, and intranasal. Parenteral infusions include intramuscular, intravenous (bollus or slow drip), intraarterial, intraperitoneal, intrathecal or subcutaneous administration.

The anti-CD47 agent need not be, but is optionally formulated with one or more agents that potentiate activity, or that otherwise increase the therapeutic effect. These are generally used in the same dosages and with administration routes as used hereinbefore or about from 1 to 99% of the heretofore employed dosages.

An anti-CD47 agent is often administered as a pharmaceutical composition comprising an active therapeutic agent and another pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

In still some other embodiments, pharmaceutical compositions can also include large, slowly metabolized macromolecules such as proteins, polysaccharides such as chitosan, polylactic acids, polyglycolic acids and copolymers (such as latex functionalized Sepharose™, agarose, cellulose, and the like), polymeric amino acids, amino acid copolymers, and lipid aggregates (such as oil droplets or liposomes).

A carrier may bear the agents in a variety of ways, including covalent bonding either directly or via a linker group, and non-covalent associations. Suitable covalent-bond carriers include proteins such as albumins, peptides, and polysaccharides such as aminodextran, each of which have multiple sites for the attachment of moieties. A carrier may also bear an anti-CD47 agent by non-covalent associations, such as non-covalent bonding or by encapsulation. The nature of the carrier can be either soluble or insoluble for purposes of the invention. Those skilled in the art will know of other suitable carriers for binding anti-CD47 agents, or will be able to ascertain such, using routine experimentation.

Acceptable carriers, excipients, or stabilizers are non-toxic to recipients at the dosages and concentrations employed, and include buffers such as phosphate, citrate, and other organic acids; antioxidants including ascorbic acid and methionine; preservatives (such as octadecyidimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as TWEEN™, PLURONICS™ or polyethylene glycol (PEG). Formulations to be used for in vivo administration must be sterile. This is readily accomplished by filtration through sterile filtration membranes.

The active ingredients may also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Carriers and linkers specific for radionuclide agents include radiohalogenated small molecules and chelating compounds. A radionuclide chelate may be formed from chelating compounds that include those containing nitrogen and sulfur atoms as the donor atoms for binding the metal, or metal oxide, radionuclide.

Radiographic moieties for use as imaging moieties in the present invention include compounds and chelates with relatively large atoms, such as gold, iridium, technetium, barium, thallium, iodine, and their isotopes. It is preferred that less toxic radiographic imaging moieties, such as iodine or iodine isotopes, be utilized in the methods of the invention. Such moieties may be conjugated to the anti-CD47 agent through an acceptable chemical linker or chelation carrier. Positron emitting moieties for use in the present invention include ¹⁸F, which can be easily conjugated by a fluorination reaction with the anti-CD47 agent.

Typically, compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the anti-CD47 agents can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in further optimizing a therapeutic dosage range for use in humans. The exact formulation, route of administration and dosage can be chosen by the individual physician in view of the patient's condition.

Suitable administration of an anti-CD47 agent (e.g., a therapeutically effective dose) can entail administration of a single dose, or can entail administration of doses daily, semi-weekly, weekly, once every two weeks, once a month, annually, etc. Dosage and frequency may vary depending on the half-life of the anti-CD47 agent in the patient. It will be understood by one of skill in the art that such guidelines will be adjusted for the molecular weight of the active agent, e.g. in the use of antibody fragments, in the use of antibody conjugates, in the use of SIRPα reagents, in the use of soluble CD47 peptides etc. The dosage may also be varied for localized administration, e.g. intranasal, inhalation, etc., or for systemic administration, e.g. i.m., i.p., i.v., and the like.

For more information on administering an anti-CD47 agent, see patent application U.S. Ser. No. 14/769,069 (Methods for Achieving Therapeutically Effective Doses of anti-CD47 Agents), which is hereby incorporated by reference in its entirety.

### Kits

Also provided are kits for use in the methods. The subject kits include a tool (e.g., a PCR primer pair specific for a SIRPα linked SNP, an antibody that specifically binds to SIRPα, and the like) for determining the level of expression. The subject kits can also include an anti-CD47 agent. An anti-CD47 agent can be provided in a dosage form (e.g., a therapeutically effective dosage form). In some embodiments, an anti-CD47 agent is provided in two or more different dosage forms (e.g., two or more different therapeutically effective dosage forms). In the context of a kit, an anti-CD47 agent can be provided in liquid or solid form in any convenient packaging (e.g., stick pack, dose pack, etc.).

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### Utility

The term “cancer”, as used herein, refers to a variety of conditions caused by the abnormal, uncontrolled growth of cells. Cells capable of causing cancer, referred to as “cancer cells”, possess characteristic properties such as uncontrolled proliferation, immortality, metastatic potential, rapid growth and proliferation rate, and/or certain typical morphological features. A cancer can be detected in any of a number of ways, including, but not limited to, detecting the presence of a tumor or tumors (e.g., by clinical or radiological means), examining cells within a tumor or from another biological sample (e.g., from a tissue biopsy), measuring blood markers indicative of cancer, and detecting a genotype indicative of a cancer. However, a negative result in one or more of the above detection methods does not necessarily indicate the absence of cancer, e.g., a patient who has exhibited a complete response to a cancer treatment may still have a cancer, as evidenced by a subsequent relapse.

The term “cancer” as used herein includes carcinomas, (e.g., carcinoma in situ, invasive carcinoma, metastatic carcinoma) and pre-malignant conditions, i.e. neomorphic changes independent of their histological origin. The term “cancer” is not limited to any stage, grade, histomorphological feature, invasiveness, aggressiveness or malignancy of an affected tissue or cell aggregation. In particular stage 0 cancer, stage I cancer, stage II cancer, stage III cancer, stage IV cancer, grade I cancer, grade II cancer, grade III cancer, malignant cancer and primary carcinomas are included.

Cancers and cancer cells that can be treated include, but are not limited to, hematological cancers, including leukemia, lymphoma and myeloma, and solid cancers, including for example tumors of the brain (glioblastomas, medulloblastoma, astrocytoma, oligodendroglioma, ependymomas), carcinomas, e.g. carcinoma of the lung, liver, thyroid, bone, adrenal, spleen, kidney, lymph node, small intestine, pancreas, colon, stomach, breast, endometrium, prostate, testicle, ovary, skin, head and neck, and esophagus; sarcomas, melanomas; myelomas; etc.

In an embodiment, the cancer is a hematological cancer. In an embodiment, the hematological cancer is a leukemia. In another embodiment, the hematological cancer is a myeloma. In an embodiment, the hematological cancer is a lymphoma.

Of interest are non-Hodgkin's lymphomas (NHLs), which are a heterogeneous group of lymphoproliferative malignancies with differing patterns of behavior and responses to treatment. Like Hodgkin's disease, NHL usually originates in lymphoid tissues and can spread to other organs, however, NHL is much less predictable than Hodgkin's disease and has a far greater predilection to disseminate to extranodal sites. The NHLs can be divided into 2 prognostic groups: the indolent lymphomas and the aggressive lymphomas. Indolent NHL types have a relatively good prognosis, with median survival in the range of 10 years, but they usually are not curable in advanced clinical stages. The aggressive type of NHL has a shorter natural history. A number of these patients can be cured with intensive combination chemotherapy regimens, but there is a significant number of relapses, particularly in the first 2 years after therapy.

Among the NHL are a variety of B-cell neoplasms, including precursor B-lymphoblastic leukemia/lymphoma; peripheral B-cell neoplasms, e.g. B-cell chronic lymphocytic leukemia; prolymphocytic leukemia; small lymphocytic lymphoma; mantle cell lymphoma; follicle center cell lymphoma; marginal zone B-cell lymphoma; splenic marginal zone lymphoma; hairy cell leukemia; diffuse large B-cell lymphoma; T-cell rich B-cell lymphoma, Burkitt's lymphoma; high-grade B-cell lymphoma, (Burkitt-like); etc.

Follicular lymphoma comprises 70% of the indolent lymphomas reported in American and European clinical trials. Most patients with follicular lymphoma are over age 50 and present with widespread disease at diagnosis. Nodal involvement is most common, often accompanied by splenic and bone marrow disease. The vast majority of patients with advanced stage follicular lymphoma are not cured with current therapeutic options, and the rate of relapse is fairly consistent over time, even in patients who have achieved complete responses to treatment. Subtypes include follicular small cleaved cell (grade 1) and follicular mixed small cleaved and large cell (grade 2). Another subtype of interest is follicular large cell (grade 3 or FLC) lymphoma which can be divided into grades 3a and 3b.

Among the aggressive forms of NHL is diffuse large B-cell lymphoma, which is the most common of the non-Hodgkin's lymphomas, comprising 30% of newly-diagnosed cases. Most patients present with rapidly enlarging masses, often with symptoms both locally and systemically. Relapses after treatment are not uncommon, depending on the presence of various risk factors. Lymphomatoid granulomatosis is an EBV positive large B-cell lymphoma with a predominant T-cell background. The histology shows association with angioinvasion and vasculitis, usually manifesting as pulmonary lesions or paranasal sinus involvement.

Mantle cell lymphoma is found in lymph nodes, the spleen, bone marrow, blood, and sometimes the gastrointestinal system (lymphomatous polyposis). Mantle cell lymphoma is characterized by CD5-positive follicular mantle B cells, a translocation of chromosomes 11 and 14, and an overexpression of the cyclin D1 protein. The median survival is significantly shorter (3-5 years) than that of other lymphomas, and this histology is now considered to be an aggressive lymphoma. A diffuse pattern and the blastoid variant have an aggressive course with shorter survival, while the mantle zone type may have a more indolent course. Refractoriness to chemotherapy is a usual feature.

Lymphoblastic lymphoma is a very aggressive form of NHL. It often occurs in young patients, but not exclusively. It is commonly associated with large mediastinal masses and has a high predilection for disseminating to bone marrow and the central nervous system (CNS). Treatment is usually patterned after that for acute lymphoblastic leukemia (ALL). Since these forms of NHL tend to progress so quickly, combination chemotherapy is instituted rapidly once the diagnosis has been confirmed. Careful review of the pathologic specimens, bone marrow aspirate and biopsy specimen, cerebrospinal fluid cytology, and lymphocyte marker constitute the most important aspects of the pretreatment staging work-up.

Burkitt's lymphoma/diffuse small noncleaved cell lymphoma typically involves younger patients and represents the most common type of pediatric non-Hodgkin's lymphoma. These aggressive extranodal B-cell lymphomas are characterized by translocation and deregulation of the c-myc gene on chromosome 8. A subgroup of patients with dual translocation of c-myc and bcl-2 appear to have an extremely poor outcome despite aggressive therapy. Treatment of Burkitt's lymphoma/diffuse small noncleaved cell lymphoma involves aggressive multidrug regimens similar to those used for the advanced-stage aggressive lymphomas.

Acute lymphocytic leukemia (ALL) generally has an aggressive course, depending in part on the presence of the Philadelphia (Ph) chromosome. Patients with Ph chromosome-positive ALL are rarely cured with chemotherapy. Many patients who have molecular evidence of the bcr-abl fusion gene, which characterizes the Ph chromosome, have no evidence of the abnormal chromosome by cytogenetics.

Hodgkin's lymphoma, is a lymphoma characterized by a pleomorphic lymphocytic infiltrate with malignant multinucleated giant cells. Most cases of Hodgkin's disease probably arise from germinal center B cells that are unable to synthesize immunoglobulin. The majority of cases in developing countries and about one third of those in the United States are associated with the presence of Epstein-Barr virus in the Reed-Stemberg cells. Treatment strategies depend on a number of factors including the presence of B symptoms, the histologic subtype, gender, and sexual maturity. To date there are several published studies demonstrating the effectiveness of Rituxan for CD20-positive Hodgkin's disease, particularly the lymphocyte predominant variant.

In an embodiment, the leukemia is selected from acute myeloid leukemia (AML), acute lymphocytic leukemia (ALL), chronic lymphocytic leukemia (CLL) and chronic myelogenous leukemia (CML). In an embodiment, the leukemia is AML. In an embodiment, the leukemia is ALL. In an embodiment, the leukemia is CLL. In a further embodiment, the leukemia is CML. In an embodiment, the cancer cell is a leukemic cell, for example, but not limited to, an AML cell, an ALL cell, a CLL cell or a CML cell.

Suitable cancers that can be responsive to treatment using an anti-CD47 agent include without limitation leukemia; acute myeloid leukemia (AML); acute lymphoblastic leukemia (ALL); metastasis; minimal residual disease; solid tumor cancers, e.g., breast, bladder, colon, ovarian, glioblastoma, leiomyosarcoma, and head & neck squamous cell carcinomas; etc. For examples, see: (i) Willingham et al., Proc Natl Acad Sci USA. 2012 Apr. 24; 109(17):6662-7: “The CD47-signal regulatory protein alpha (SIRPα) interaction is a therapeutic target for human solid tumors”; (ii) Edris et al., Proc Natl Acad Sci USA. 2012 Apr. 24; 109(17):6656-61: “Antibody therapy targeting the CD47 protein is effective in a model of aggressive metastatic leiomyosarcoma”; and (iii) US patent application 20110014119; all of which are herein incorporated in their entirety.

The invention now being fully described, it will be apparent to one of ordinary skill in the art that various changes and modifications can be made without departing from the spirit or scope of the invention.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Materials and Methods

Primary Human Samples. Peripheral Blood Samples and Leukoreduction System (LRS) Chambers were obtained from the Stanford Blood Center. Peripheral Blood Mononuclear Cells (PBMCs) were isolated by Ficoll density centrifugation using standard methods. PMBCs were analyzed immediately for basal expression of indicated cell markers by flow cytometry.

SNP genotyping. DNA was isolated from frozen cell pellets of PBMCs or monocytes from each donor using PureLink Genomic DNA Mini Kit (Invitrogen). Primers used to amplify the region containing SNP rs4813322 were as follows: TCCGGAAAGTTCCTAGCTCA (forward) and GGTTCTCATGGCAGGTGAGT (reverse). The PCR product was validated by gel electrophoresis and purified with Qiagen PCR Purification kit. Purified PCR products were submitted for Sanger Sequencing and sequence traces used to identify the genotype of each donor.

Monocyte-derived macrophages. Monocytes were further purified from PBMCs through Percoll density centrifugation and differentiated to macrophages by 7-10 days of culture in IMDM+GlutaMax (Life Technologies) supplemented with 10% AB Human Serum (Life Technologies) and 100 U/ml penicillin and streptomycin (Life Technologies).

T Cell Activation. Wells of multi-well tissue culture plates were coated with PBS or 1 μg/ml purified anti-human CD3 and 3 μg/ml purified anti-human CD28 antibodies (BioLegend) for 2 hours at 37° C. Wells were then washed three times with PBS. PBMCs were pulsed with CFSE (Thermo) for 15 minutes at 37° C., washed and cultured in duplicates for each condition in RPMI supplemented with 10% FBS, 1× GlutaMAX, 25 mM HEPES, 100 U/ml penicillin, 100 μg/ml streptomycin, 1×MEM Non-Essential Amino Acids, 1 mM sodium pyruvate (Life Technologies) and 50 U/ml rhIL-2 (Peprotech). Cells were analyzed by flow cytometry on day 4.

Chimeric antigen receptors (CAR) T cell Generation and cytotoxicity assay. Constructs for CD19.28C CAR and its production were previously described (see Long et al. Nat. Med. 21:581-590 (2015)). T cells were isolated from peripheral blood using RosetteSep Human T cell Enrichment Cocktail (STEMCELL) and activated with anti-CD3/CD28 beads (Life Technologies) for 3 days. On days 3 and 4, activated T cells were retrovirally transduced and cultured in AIM-V (Life Technologies) supplemented with 5% FBS, 10 mM HEPES, 100 U/ml penicillin, 100 μg/ml streptomycin, 2 mM L-glutamine and rhIL-2 (Prometheus). T cells were analyzed by FACS on day 9 and cytotoxicity assays were carried out on day 10. GFP+ Nalm6 tumor cell line expressing low levels of CD19 was used as targets and co-incubated with CD19 CAR T cells at a 1:1 ratio in the presence of 10 μg/ml isotype control antibody or anti-SIRPα antibody (KWAR). Each condition was carried out in triplicates in a 96-well flat bottom tissue cell plate. GFP signal was captured periodically during a span of 120 hours using the IncuCyte Live Imaging System. Normalized GFP intensity is calculated for each well at each recorded timepoint by dividing the average GFP signal at that timepoint by that at time zero.

Phagocytosis Assays. GFP+ MCF7 cells were incubated with MDMs at a ratio of 2:1 in IMDM without serum at 37° C. for 2 hours, in the presence of 10 μg/ml per reaction well of anti-CD47 antibody or isotype control. Phagocytosis reactions were quenched by the addition of ice cold PBS and reactions were stained with conjugated anti-CD11b antibody to label macrophages and anti-SIRPα antibody (SESA5) to measure macrophage SIRPα expression. Reactions were analyzed using flow cytometry to quantify phagocytosis events, as defined by CD11b+ macrophages also positive for the cancer cell fluorescent label (GFP). Phagocytosis fold change was calculated by dividing percent of macrophages that phagocytose with anti-CD47 antibody treatment by that with isotype control treatment.

Flow Cytometry Analysis. Antibodies targeting the following markers were used for analysis of freshly isolated PBMCs and activated T cells: CD3, CD4, CD8, CD14, HLA-DR, CD141, CD11c, CD172a (clone SESA5) and CD163. Additionally, anti-CD11b antibody was used for analysis of monocyte-derived macrophages. All antibodies were purchased from BioLegend unless otherwise stated.

Additional SNPs associated with altered SIRPα expression are listed in Table 1. The data was obtained from the eQTLgen consortium. The disclosed effect is on SIRPA gene expression. Each assessed allele A is associated with altered expression transcriptionally. The z score is the expression difference between two alleles corresponding to the number of standard deviations apart, where a negative Z score indicate decreased expression of SIRPα. Alleles that are associated with the highest impact, having a z-score around 21 standard deviations, are correlated with each other and appear in linkage disequilibrium. Any single one of these linked SNPs can be used for prognosis.

TABLE 1
SNPs on Chromosome 20 associated with altered SIRPa Expression

| ID | Pos (hg19) | Pos (hg19) | Z-score | Assessed | Other |
|---|---|---|---|---|---|
| rs6132062 | 1896401 | 1897848 | −21.6135 | A | T |
| rs6075340 | 1894335 | 1897848 | −21.6074 | A | G |
| rs6136376 | 1896244 | 1897848 | −21.5977 | G | A |
| rs6136377 | 1896288 | 1897848 | −21.531 | G | A |
| rs4813330 | 1897407 | 1897848 | −21.4647 | G | A |
| rs6075339 | 1894315 | 1897848 | −21.4469 | T | C |
| rs4813323 | 1893815 | 1897848 | −21.4285 | A | G |
| rs4813331 | 1897442 | 1897848 | −21.4064 | T | C |
| rs6081121 | 1889858 | 1897848 | −21.3988 | G | A |
| rs59317774 | 1888865 | 1897848 | −21.3772 | G | A |
| rs1569960 | 1893121 | 1897848 | −21.371 | A | G |
| rs72620874 | 1895889 | 1897848 | −21.2737 | C | G |
| rs6045331 | 1885099 | 1897848 | −21.2304 | G | C |
| rs4813322 | 1893809 | 1897848 | −21.2261 | A | G |
| rs4813324 | 1893980 | 1897848 | −21.0866 | C | G |
| rs4813325 | 1893986 | 1897848 | −21.0828 | G | C |
| rs6081134 | 1894467 | 1897848 | −21.0433 | A | T |
| rs4813321 | 1893784 | 1897848 | −21.0315 | A | G |
| rs4813319 | 1893747 | 1897848 | −21.0183 | C | T |
| rs4813320 | 1893763 | 1897848 | −20.9906 | G | C |
| rs17775933 | 1894977 | 1897848 | −20.9822 | G | A |
| rs68064046 | 1895016 | 1897848 | −20.9611 | A | G |
| rs16978863 | 1894630 | 1897848 | −20.9066 | A | G |
| rs66815411 | 1894789 | 1897848 | −20.8465 | C | T |
| rs68178619 | 1894872 | 1897848 | −20.7904 | C | T |
| rs67414469 | 1895273 | 1897848 | −20.7726 | C | T |
| rs4813329 | 1897390 | 1897848 | −20.6586 | G | C |
| rs55698111 | 1894584 | 1897848 | −20.363 | C | T |
| rs111751479 | 1895644 | 1897848 | −19.7842 | C | T |
| rs113347998 | 1895655 | 1897848 | −19.7112 | C | T |
| rs72620873 | 1895533 | 1897848 | −19.6693 | C | T |
| rs113961013 | 1895658 | 1897848 | −19.5678 | T | A |
| rs67774686 | 1895071 | 1897848 | −18.6109 | T | C |
| rs67343776 | 1894920 | 1897848 | −18.5541 | C | T |
| rs72620870 | 1894896 | 1897848 | −18.3259 | G | T |
| rs67558779 | 1894847 | 1897848 | −18.2933 | G | C |
| rs66675914 | 1895292 | 1897848 | −18.2847 | G | A |
| rs67772748 | 1894910 | 1897848 | −18.2307 | C | G |
| rs72620869 | 1894881 | 1897848 | −18.1946 | C | T |
| rs66600581 | 1895601 | 1897848 | −18.193 | T | C |
| rs56301259 | 1894606 | 1897848 | −18.174 | T | C |
| rs112203261 | 1895621 | 1897848 | −18.103 | A | C |
| rs66523711 | 1895570 | 1897848 | −18.0921 | T | C |
| rs111980944 | 1895630 | 1897848 | −18.0719 | C | T |
| rs66591681 | 1895317 | 1897848 | −18.0207 | T | C |
| rs8116907 | 1869347 | 1897848 | 17.8713 | C | T |
| rs55797358 | 1894690 | 1897848 | −17.8371 | A | T |
| rs56380582 | 1894689 | 1897848 | −17.8311 | T | G |
| rs56278099 | 1894607 | 1897848 | −17.7684 | G | A |
| rs2318123 | 1871972 | 1897848 | 17.7472 | A | T |
| rs156355 | 1818613 | 1897848 | 17.5749 | C | T |
| rs4814731 | 1889201 | 1897848 | −17.4505 | T | C |
| rs548475 | 1832989 | 1897848 | 16.7935 | T | C |
| rs4813309 | 1880550 | 1897848 | −16.7729 | T | C |
| rs156343 | 1834545 | 1897848 | 16.7728 | G | A |
| rs17853847 | 1895842 | 1897848 | −16.7696 | C | T |
| rs156350 | 1839408 | 1897848 | 16.7586 | C | T |
| rs73569345 | 1895611 | 1897848 | −16.7455 | A | G |
| rs156351 | 1839442 | 1897848 | 16.7291 | G | A |
| rs156352 | 1840343 | 1897848 | 16.6852 | C | T |
| rs17855612 | 1895835 | 1897848 | −16.6125 | T | C |
| rs156333 | 1827655 | 1897848 | 16.5657 | A | G |
| rs11698383 | 1861882 | 1897848 | 16.564 | G | C |
| rs156328 | 1825326 | 1897848 | 16.5376 | T | C |
| rs156332 | 1827015 | 1897848 | 16.5273 | A | G |
| rs6081096 | 1884290 | 1897848 | −16.4904 | C | G |
| rs156325 | 1847540 | 1897848 | 16.4194 | C | T |
| rs156327 | 1824234 | 1897848 | 16.3993 | T | C |
| rs6080978 | 1862420 | 1897848 | 16.3817 | T | C |
| rs156330 | 1826095 | 1897848 | 16.3719 | T | G |
| rs156324 | 1846014 | 1897848 | 16.3001 | C | T |
| rs6136293 | 1862614 | 1897848 | 16.2808 | C | T |
| rs6035018 | 1882954 | 1897848 | −16.25 | C | T |
| rs4814734 | 1889477 | 1897848 | −16.2472 | A | T |
| rs149853 | 1822324 | 1897848 | 16.1847 | C | G |
| rs627517 | 1821917 | 1897848 | 16.1094 | A | C |
| rs156338 | 1854678 | 1897848 | 15.9983 | T | C |
| rs156334 | 1827809 | 1897848 | 15.9298 | T | C |
| rs156329 | 1825944 | 1897848 | 15.6645 | T | C |
| rs552067 | 1823326 | 1897848 | 15.654 | G | C |
| rs156356 | 1819280 | 1897848 | 15.6319 | C | T |
| rs156358 | 1819824 | 1897848 | 15.6236 | C | G |
| rs6111858 | 1866645 | 1897848 | 15.6025 | A | G |
| rs1135192 | 1895794 | 1897848 | −15.5687 | A | G |
| rs156342 | 1834194 | 1897848 | 15.5586 | T | C |
| rs55646123 | 1834992 | 1897848 | 15.5556 | G | C |
| rs149222 | 1834907 | 1897848 | 15.5482 | A | G |
| rs149221 | 1835001 | 1897848 | 15.5469 | G | A |
| rs156344 | 1835757 | 1897848 | 15.5147 | A | G |
| rs156345 | 1835786 | 1897848 | 15.5124 | G | A |
| rs156346 | 1836361 | 1897848 | 15.489 | C | T |
| rs156341 | 1834059 | 1897848 | 15.4622 | G | A |
| rs156347 | 1836532 | 1897848 | 15.4415 | G | A |
| rs156348 | 1837509 | 1897848 | 15.4094 | G | A |
| rs146490405 | 1895797 | 1897848 | −15.4053 | A | G |
| rs7273343 | 1832855 | 1897848 | 15.3719 | T | A |
| rs113123564 | 1830589 | 1897848 | 15.3361 | T | C |
| rs156323 | 1845184 | 1897848 | 15.1954 | A | T |
| rs17855610 | 1895820 | 1897848 | −15.1272 | T | C |

1. A method of determining whether a cell or cell population is responsive to therapeutic CD47 blockade, the method comprising:
assaying a cell sample from an individual to determine (a) the level of expression of SIRPα on phagocytic immune cells, or (b) the genotype of the individual at an SNP associated with SIRPα expression;
determining that the individual is responsive when expression levels of SIRPα is predicted to be low.

2. The method of claim 1, wherein the SNP associated with SIRPα expression is selected from the SNP of Table 1, rs4813322 or a SNP linked to rs4813322.

3. The method of claim 1, where the genotype of the individual at SNP rs4813322 is determined and wherein an individual that is heterozygous G/G or heterozygous A/G at the SNP is predicted to be more responsive than an individual that is heterozygous A/A at the SNP.

4. The method of claim 1, wherein the genotype of the individual at one or more SNPs linked to SNP rs4813322 is determined, wherein an SN linked to heterozygous G/G or heterozygous A/G at the rs4813322 is predicted to be more responsive than an individual that is heterozygous A/A at rs4813322.

5. The method of claim 1, wherein genotyping is performed by sequencing.

6. The method of claim 1, wherein genotyping is performed by hybridization.

7. The method of claim 1, wherein genotyping is performed by sequence specific amplification.

8. The method of claim 1, wherein genotyping is performed by single strand conformational polymorphism analysis.

9. The method of claim 1, wherein basal or induced SIRPα expression is determined for in any of dendritic cells, monocytes, macrophages and T cells, including engineered T cells such as CAR T cells.

10. The method of claim 9, wherein measuring comprises measuring protein levels of SIRPα.

11. The method of claim 9, wherein the assaying steps comprise measuring the level of mRNA of SIRPα.

12. The method of claim 1 wherein the biological sample is one or more of a swab, skin sample, blood sample, a biopsy sample, a fine needle aspirate.

13. The method of claim 1, further comprising the step of administering an anti-CD47 agent to the individual determined to be responsive to an anti-CD47 agent.

14. The method of claim 1, further comprising:
providing an analysis indicating whether the individual is determined to be responsive or not responsive to the anti-CD47 agent.
